(19) United States
(12) Patent Application Publication
(10) Pub. No.: US 2009/0232848 A1
(43) Pub. Date: Sep. 17, 2009

(54) METHODS FOR CULTIVATING LAWSONIA INTRACELLULARIS

(75) Inventor: Srinivas Saginala, Kalamazoo, MI (US)

Correspondence Address: PHARMACIA & UPJOHN, 7000 Portage Road, KZO-300-104, KALAMAZOO, MI, 49001, US

(73) Assignee: PFIZER INC., New York, NY (US)

(21) Appl. No.: 11/719,884

(22) PCT Filed: Nov. 14, 2005
(86) PCT No.: PCT/IB05/03496

§ 371 Date: Jun. 5, 2007

Related U.S. Application Data
(60) Provisional application No. 60/630,728, filed on Nov. 24, 2004.

(51) Int. Cl. A61K 39/02; C12N 1/20; G01N 33/53; C12Q 1/68; A61P 31/04

(52) U.S. Cl. 424/234.1; 435/252.1; 435/7.1; 435/6

(57) ABSTRACT

This invention relates to methods for cultivating Lawsonia intracellularis. In particular, the present invention provides improved methods for cultivating Lawsonia intracellularis by employing reducing agents other than molecular hydrogen; or alternatively, by employing a combination of one or more reducing agents with molecular hydrogen. This invention also relates to vaccines and diagnostic reagents prepared from Lawsonia intracellularis cultivated by employing the methods disclosed herein.

### FIELD OF THE INVENTION

This invention relates to methods for cultivating Lawsonia intracellularis. This invention also relates to vaccines and diagnostic reagents prepared from Lawsonia intracellularis cultivated in accordance with the methods disclosed herein.

### BACKGROUND OF THE INVENTION

Hydrogen gas is hydrogen in the form of a gas or in solution and is referred to as “H₂” or as “molecular hydrogen” or as “molecular H₂”. It may be in the form of a gas or dissolved in or introduced into a solution. Non-molecular hydrogen is any bound form of H such as are found in organic or inorganic reducing agents, examples are provided.

Inorganic reducing agents are any chemical reducing agents without a carbon nucleous, non limiting examples of such agents are: hydrosulfite (dithionite), thiosulfate, disulfite (metabisulfite), hydrogen sulfide and free base forms, hydrochlorides, hydrates, and salts thereof.

Lawsonia intracellularis is the pathogen that causes porcine proliferative enteropathy (PPE). The organism has also been previously referred to as “Campylobacter-like” organism (McOrist et al., Vet. Pathol. 26: 260-264, 1989), and as “Ileal symbiont intracellularis” (Stills, Infection & Immunol. 59: 3227-3236, 1991).

Lawsonia intracellularis is an obligate intracellular bacterium which cannot be cultured by normal bacteria cultivation methods using conventional cell-free media. It can only be cultured in vitro with tissue culture cells (Joens et al., Am. J. Vet. Res. 58: 1125-1131,1997; Lawson et al., J. Clinical Microbiology 31: 1136-1142, 1993; McOrist, Int. J. Systematic Bacteriology 45: 820-825, 1995; International Patent Application PCT/US96/09576).

In infected animals, L. intracellularis is located in the cytoplasm of the villus cells and intestinal crypt cells. Pigs suffering from PPE are characterized by irregularities in the villus cells and intestinal crypt structure with epithelial cell dysplasia, wherein crypt abscesses form as the villi and intestinal crypts become branched and fill with inflammatory cells.

Organic reducing agents are any chemical reducing agents containing carbon, non limiting examples of such agents are: the group consisting of alkyl thiols, aryl thiols, L-cysteine, D-cysteine, homocysteine, β-mercaptoethanol, ethanethiol, propanethiol, dithiothreitol, cysteamine, cysteine persulfides, glutathione, dimercaptosuccinic acid (DMSA), tris(2-carboxyethyl) phosphine hydrochloride (TCEP), tributylphosphine (TBP), and enantiomers, racemic forms or mixtures, free base forms, hydrochlorides, hydrates, and salts thereof.

PPE is a disease of commercial significance to the swine industry. PPE is associated with stock losses, medication costs, reduced growth rates of pigs and increased feed costs. PPE also contributes to downstream indirect costs in, for example, additional labor costs and environmental costs in dealing with antibiotic residue contamination, and in control measures to prevent the organism from being passed on or carried to other animals or humans.

Reducing agents are any chemical compounds that can act as hydrogen donors.

Reducing agents have reducing strengths referred to as redox potentials according to their strength and how much is used. Here the range of redox potentials considered especially useful are: +300 mV to −600 mV (mV is milliVolt), more preferred is +100 mV to −400 mV and most preferred is −100 mV to −300 mV. The strength of the reducing agent can also be referred to by the concentration of the agent. Here the concentration ranges, considered especially useful are:, when used as a percent, is 0.8% to 0.0008%, more preferred is 0.4% to 0.004%, more preferred is 0.10% to 0.002% and most preferred 0.02 to 0.002%. Here the concentration ranges of the reducing agent can also be thought of in milliMolar (mM), considered especially useful concentration ranges are: 0.05 mM to 50.0 mM, more preferred is 0.10 mM to 10.0 mM and most preferred is 0.10 mM to 2.0 mM. Redox potentials, and concentrations will vary depending on other reducing or oxidizing agents in solution, determining optimal levels of what reducing agents to use and what redox potentials or concentrations should be a matter of routine for one skilled in the art.

The difficulty in cultivating L. intracellularis, even under the optimal conditions so far identified, remains an obstacle to the understanding and control of PPE. There is a need for improved and safer methods for cultivating Lawsonia intracellularis, and for the development of compositions for treating and preventing PPE.

### SUMMARY OF THE INVENTION

The present invention is directed to methods of cultivating Lawsonia intracellularis.

In one embodiment, the present invention provides a method for cultivation of L. intracellularis in the absence of molecular hydrogen, H₂.

In a specific embodiment of the present invention, one or more organic or inorganic reducing agents, other than molecular hydrogen, are employed in tissue culture medium for enhanced cultivation of L. intracellularis.

In a preferred embodiment, tissue culture medium for cultivating L. intracellularis is supplemented with cysteine or a salt thereof.

In another embodiment, the present invention provides a method for cultivation of L. intracellularis wherein tissue culture medium is supplemented with both molecular hydrogen and one or more organic or inorganic reducing agents other than molecular hydrogen.

In a specific embodiment, tissue culture medium is supplemented with both molecular hydrogen and cysteine or a salt thereof.

In still another embodiment, the present invention provides a method for cultivation of L. intracellularis at near ambient and enriched oxygen concentrations.

In a specific embodiment, L. intracellularis is cultivated under near ambient and enriched oxygen concentrations in the presence of one or more organic or inorganic reducing agents, and in the absence of molecular hydrogen.

In another specific embodiment, L. intracellularis is cultivated under near ambient and enriched oxygen concentrations in the presence of molecular hydrogen and one or more organic or inorganic reducing agents other than molecular hydrogen.

L. intracellularis cultivated in accordance with the methods of the present invention can be employed in the production of L. intracellularis vaccines and diagnostic reagents. Accordingly, vaccines and diagnostic reagents prepared from L. intracellularis cultivated with the methods described herein form another embodiment of the present invention.

This invention discloses a novel method for cultivating Lawsonia intracellularis, the method comprising the use of chemical reducing agents either with or without molecular H₂. The method can be used to either reduce the levels of H gas commonly used to grow Lawsonia intracellularis, or they can be used to eliminate the use of the addition of molecular H₂. Examples and details are provided. Useful related vaccines, effective in treating or preventing a disease in an animal caused by L. intracellularis comprising an immunologically effective amount of L. intracellularis grown by any of the methods, optionally containing an adjuvant, optionally used to treat a pig, methods of diagnosing a disease in comprising detecting the presence of antibodies in a sample from said animal that are reactive with or with an antibody generated, or a a polynucleotide isolated from the L. intracellularis cultured according to the procedures and a A kit are provided. Also provided are method for cultivating Lawsonia intracellularis wherein the cells are cultured at an O₂ concentration in the range of about 2% to 18%.

The reducing agent other than other than molecular H₂ may be an organic or inorganic reducing agent with a redox potential range of +300 mV to −600 mV. It may be an organic reducing agent is selected from the group consisting of: alkyl thiols, aryl thiols, L-cysteine, D-cysteine, homocysteine, β-mercaptoethanol, ethanethiol, propanethiol, dithiothreitol, cysteamine, cysteine persulfides, glutathione, dimercaptosuccinic acid (DMSA), tris(2-carboxyethyl) phosphine hydrochloride (TCEP), tributylphosphine (TBP), and enantiomers, racemic forms or mixtures, free base forms, hydrochlorides, hydrates, and salts thereof. It may be an inorganic reducing agent, wherein said inorganic reducing agent is selected from the group consisting of: hydrosulfite (dithionite), thiosulfate, disulfite (metabisulfite), hydrogen sulfide and free base forms, hydrochlorides, hydrates, and salts thereof.

The concentration of said reducing agents are selected from the following ranges: the range of redox potentials are about: +300 mV to −600 mV (mV is milliVolt), more preferred is +100 mV to −400 mV and most preferred is −100 mV to −300 mV, alternatively the reducing agent concentration is in the following ranges, about: 0.8% to 0.0008%, more preferred is 0.4% to 0.004%, more preferred is 0.10% to 0.002% and most preferred 0.02 to 0.002%, alternatively the concentration ranges in milliMolar (mM), considered especially useful concentration ranges are, about: 0.05 mM to 50.0 mM, more preferred is 0.10 mM to 10.0 mM and most preferred is 0.10 mM to 2.0 mM

### DETAILED DESCRIPTION OF THE INVENTION

Prior to the present invention, it was generally understood that the cultivation of Lawsonia intracellularis in tissue culture cells required the addition of molecular hydrogen (H₂) to the vessel gas phase or headspace. L. intracellularis was routinely cultivated in tissue culture cells in the presence of about 73 to 94% H₂ and under reduced oxygen concentrations. In addition to the need for providing a source of hydrogen during cultivation, the use of H₂ concentrations above 4% created a potentially hazardous scenario in the laboratory.

The present inventors have discovered that reducing agents other than molecular hydrogen permit cultivation of L. intracellularis in the absence of molecular hydrogen. That these reducing agents are at least as potent as molecular hydrogen in promoting propagation of L. intracellularis is unexpected. Additionally, a combination of one or more of these reducing agents with molecular hydrogen may enhance cultivation of L. intracellularis. Accordingly, the present invention provides safer and more economic methods for cultivating L. intracellularis.

The term “cultivating” or “cultivation”, as used herein, refers to the process of promoting the growth, reproduction and/or proliferation of L. intracellularis in tissue culture cells.

Generally speaking, tissue culture cells are first infected with an inoculum of L. intracellularis bacteria. Cells suitable for use in cultivating L. intracellularis are known in the art (see, e.g., U.S. Pat. No. 5,714,375 and International Patent Application PCT/US01/30284), and include but are not limited to, simian cells, murine cells, rat cells, canine cells, feline cells, hamster cells, human cells, equine cells, fish cells, bovine cells and swine cells. Preferably, L. intracellularis is cultivated using rat intestinal epithelial cells IEC-18 (ATCC 1589), human epidermoid carcinoma cells HEp-2 (ATCC 23), mouse McCoy cells (ATCC 1696), Madin-Darby canine kidney cells MDCK (ATCC 34), buffalo green monkey kidney cells BGMK (Biowhittaker #71-176), swine intestinal epithelium cells, and Vero cells. Especially preferred cells are HEp2, McCoy or IEC-18 cells.

Prior to being inoculated, the cells can be cultured in conventional tissue culture flasks, bottles or chambers containing growth media. The growth media can be any commercially available media, typically including a nitrogen source, necessary growing factors for the chosen culture cells, and a carbon source, such as glucose or lactose. A preferred medium is DMEM, supplemented with 2-10% fetal bovine serum.

The inoculum can be a pure culture of L. intracellularis obtained, for example, from the American Type Culture Collection, or from infected swine or other animals using the isolation and purification techniques well known to those skilled in the art.

The inoculum is added to a cell culture to infect the cells, and the inoculated cells are then incubated under appropriate conditions. According to the present invention, the inoculated cells can be cultivated in the absence of molecular hydrogen. In particular, it has been uniquely identified in accordance with the present invention that reducing agents other than molecular hydrogen are as potent as molecular hydrogen for cultivating L. intracellularis. Therefore, inoculated cells can be cultured in the absence of molecular hydrogen and in the presence of one or more reducing agents other than molecular hydrogen to achieve sufficient growth, reproduction and proliferation of L. intracellularis.

According to the present invention, reducing agents appropriate for cultivation of L. intracellularis include, but are not limited to, reduced organic sulfur compounds such as, e.g., alkyl thiols, aryl thiols, L-cysteine, D-cysteine, homocysteine, β-mercaptoethanol, ethanethiol, propanethiol, dithiothreitol, cysteamine(2-mercaptoethylamine), cysteine persulfides, glutathione, dimercaptosuccinic acid (DMSA), other thiol-containing agents, and mixtures of reduced and oxidized thiol-containing agents (e.g. cysteine-cystine, cysteamine-cystamine); reduced inorganic sulfur compounds such as, e.g., hydrosulfite (dithionite), thiosulfate, disulfite (metabisulfite), and hydrogen sulfide; phosphine derivatives such as, e.g., tris(2-carboxyethyl)phosphine hydrochloride (TCEP) and tributylphosphine (TBP); ascorbic acid; enantiomers, racemic forms or mixtures, free base forms, hydrochlorides, hydrates, and salts of all relevant reducing agents, Oxyrase® and other enzymatically-based reducing systems, and other constituents, components, additions, or conditions capable of establishing a reducing redox environment in culture media. It is fairly convenient to supply these reducing agents to culture media. Therefore, the identification of these reducing agents by the present invention provides convenient and effective alternatives for cultivating L. intracellularis.

Further in accordance with the present invention, the inoculated cells can be cultivated in the presence of molecular hydrogen in combination with one or more reducing agents described above to achieve enhanced cultivation of L. intracellularis. By “enhanced cultivation” is meant increased propagation, viability or motility of L. intracellularis, as compared to cultivation in the presence of molecular hydrogen or a reducing agent individually.

Other important cultivation parameters include the concentration of O₂ and CO₂. Prior to the present invention, cells inoculated with L. intracellularis were typically cultivated at a reduced O₂ concentration, generally in the range of 2% to 18%; preferably in the range of from about 4% to about 10%; and more preferably, at about 8.0%. According to the present invention, the use of one or more reducing agents other than molecular hydrogen, as described above, permit cultivation of cells inoculated with L. intracellularis under near ambient and enriched O₂ concentrations, for example, 19 to 21%, and above.

Appropriate concentrations of carbon dioxide have been described in the art, e.g., in U.S. Pat. No. 5,714,375. Preferably, the inoculated cells are incubated in a carbon dioxide concentration in the range from about 6% to about 9%, with a carbon dioxide concentration of about 8.8% being most preferred.

According to the present invention, it is also preferred that inoculated cells are cultivated in the presence of nitrogen (N₂). Preferably, the inoculated cells are incubated in a N₂ concentration in the range from about 71% to about 98%; more preferably, from about 74% to about 87%, with a N₂ concentration of about 83.2% being most preferred.

In a particularly preferred embodiment, the cells are incubated in culture media supplied with cysteine hydrochloride, in an atmosphere of about 8.0% O₂, about 8.8% CO₂, and about 83.2% N₂.

In another specific embodiment, the cells are incubated in culture media supplied with cysteine hydrochloride, which media has been treated with hydrogen gassing, and in an atmosphere of about 8.0% O₂, about 8.8% CO₂, and about 83.2% N₂.

The inoculated cells are typically seeded in tissue culture flasks or bottles, which are placed in appropriate incubation devices routinely used by those skilled in the art, for example, a dual gas incubator or other gas chamber that can be easily regulated for the atmosphere and temperature within the incubator or chamber. If desired, the tissue culture flasks or bottles can be agitated to maintain the cells in a suspended state during incubation. For optimal cell growth, about 25-50% of the culture is removed and replaced with fresh media every two to three days.

To expand the production of L. intracellularis, cultivated L. intracellularis can be passaged to fresh culture cells. The passage of L. intracellularis in suspension cultures can be accomplished by removing a portion of the suspension culture of infected cells and adding it to a new flask containing fresh (i.e., uninfected) culture cells. The passage of monolayer cell cultures is achieved by lysing the cells, harvesting L. intracellularis from cell lysates, and infecting fresh cell cultures with harvested L. intracellularis.

After sufficient growth of the cultured cells, the cultivated L. intracellularis is then harvested using techniques well known to those skilled in the art. Generally speaking, the cultured cells are collected and lysed by, e.g., passing a cell suspension through a 25 gauge needle. Cellular nuclei and debris are removed from the cell lysate, and L. intracellularis can be collected from the supernatant by centrifugation. The collected L. intracellularis bacteria are suspended in appropriate diluent suitable for either passaging or formulating diagnostic reagents or vaccine compositions.

In a further aspect of the present invention, L. intracellularis cultivated by employing the present methods are used in the preparation of a diagnostic reagent. For example, the bacterial cells can be used directly as an antigen for detecting antibodies to L. intracellularis in the serum and other body fluids of animals suspected of being infected with the bacteria. Alternatively, the bacterial cells can be used to isolate polynucleotides, polypeptides, which can also be used to generate antibodies. The isolated polunucleotides, polypeptides and antibodies can then be used in diagnostic assays. The diagnostic reagent can be provided in the form of a kit.

In another aspect of the present invention, L. intracellularis cultivated in accordance with the present invention are used in formulating vaccine compositions. The bacteria can be inactivated using formalin or other inactivating agents. Alternatively, the bacteria can be attenuated by using any of the known attenuation techniques, e.g., by high serial passaging or chemical means. Inactivated or attenuated live bacteria can be combined with a suitable adjuvant, such as aluminum hydroxide or mineral oil to enhance the immunogenicity of the vaccine.

The vaccines compositions prepared from L. intracellularis cultivated in accordance with the present invention are useful for protecting animals (such as pigs, rodents, rabbits, sheep, horses, monkeys, dogs, deer, foxes, and birds), especially pigs, against a disease caused by L. intracellularis, such as PPE. Therefore, methods of treating or preventing a disease caused by L. intracellularis in an animal form another embodiment of the present invention.

The present invention is further illustrated, but not limited, by the following examples.

### EXAMPLE 1

### Initial Propagation Studies with L. intracellularis Swine Isolate VP1: Cysteine Hydrochloride Versus Hydrogen Gassing

L. intracellularis swine isolate VP1 (passage 17) was used to infect McCoy cells. McCoy cells were seeded in tissue culture flasks at 1.25×10⁵ cells per 25 cm² flask, and 1.25×10⁴ cells per Trac bottle (Bibby Sterilin Ltd.; Staffordshire, United Kingdom), in DMEM supplemented with 7% fetal bovine serum (FBS). The Trac bottle was used to monitor simulative infection in the flask. The flasks and Trac bottles were incubated overnight at 37° C. in a humidified incubator with 5% CO₂. The following day, cells in both the flask and Trac bottle were infected with the L. intracellularis swine isolate (passage 17) in fresh DMEM with 7% FBS. After the addition of L. intracellularis, the flasks and Trac bottles were treated in three different ways. One flask and the corresponding Trac bottle were evacuated at 15 psi of Hg vacuum and purged with pure hydrogen. A second flask and the corresponding Trac bottle were neither evacuated nor purged with hydrogen, but instead received a supplement of cysteine hydrochloride (Sigma, C-7477; St. Louis, Mo.), β-NAD and phoshatidylcholine to a final concentration of 0.01%, 5 mM and 100 ng/ml, respectively, during the first two passages, and only cysteine hydrochlride (0.02% final concentration) during the later passages. The third flask and the corresponding Trac bottle were not evacuated or purged with hydrogen, nor received any supplements. All flasks and Trac bottles were incubated at 37° C. in an incubator containing 8.0 % O₂ and 8.8% CO₂ and 83.2% N₂. On day 2 and 5 post-infection, 50% of the media from all flasks and Trac bottles was replaced with fresh DMEM with 5% FBS with or without cysteine hydrochloride, β-NAD and phoshatidylcholine.

On day 5 or 6 post-infection, the monolayers in the tissue culture flasks were monitored by immunoperoxidase staining of the corresponding Trac bottle cover slip. Briefly, the cover slip from the Trac bottle was removed and washed gently with Phosphate Buffered Saline (PBS), and bound to a microscope slide using a permanent glue. The cover slip then was fixed in acetone for 30 sec at room temperature. Rabbit anti-L. intracellularis polyclonal antibody, diluted 1:400 in PBS, was added to cover the entire cover slip, which was then incubated in a humidified environment at 37° C. for 30 min. The cover slip was washed gently with PBS and a 1:20 dilution of peroxidase-conjugated goat anti-rabbit IgG (H+L; Kirkegaard & Perry Laboratories, Inc.; Gaithersburg, Md.) was added to the cover slip and incubated for 30 min at 37° C. Peroxidase substrate solution was prepared by dissolving 10 mg of 3,3′-Diaminobenzidine (DAB; Sigma-Aldrich; St. Louis, Mo.) in 20 ml of PBS. After filtration through Whatman 113V filter paper (Whatman International Ltd.; Kent, United Kingdom), 40 μl of 30% H₂O₂ was added to the dissolved substrate. The final substrate solution was added to the cover slip and incubated at room temperature for 5 min. After rinsing with tap water, the cover slip was counterstained for 30 sec using Modified Harris Hematoxylin Solution (Sigma-Aldrich). The stained slide then was rinsed with tap water and observed under a microscope. Infection of the McCoy cells was also assessed by microscopic viewing of the bacteria in the media within the flask.

The infected McCoy cells in the flask were lysed using a 0.1% potassium chloride (KCl) solution on day 6 or 7 post-infection. The lysed material was used to re-infect fresh McCoy cell monolayers in flasks and Trac bottles. Briefly, after removing the media from the flask, a 0.1% KCl solution was added (5 ml per 25 cm²) and incubated at 37° C. for 5-10 min. The KCl solution was removed from the flask, and 2 ml of sucrose potassium glutamate (SPG; 0.218M sucrose, 0.0038M KH₂PO₄, 0.0072M K₂H PO₄, and 0.0049M potassium glutamate) with 5% FBS was added. A cell scraper was then used to remove the cells from the flask. They were then lysed by forcing them through a syringe fitted with a 18 ga needle. An aliquot of the lysed material was observed under a microscope to ensure satisfactory lysis. The lysed material and the supernatant removed from the flask prior to lysis were centrifuged at 3500 g for 15-20 min. The resultant pellet was resuspended in the appropriate volume of SPG with 5% FBS, depending on the number of bacteria. An appropriate amount of DMEM with 5% FBS was added to the resuspended cell lysate and used to re-infect fresh McCoy cell monolayers as described earlier.

Based on the results of these experiments (Table 1), it was concluded that the infection of McCoy cells with L. intracellularis required the use of hydrogen or supplementation with cysteine HCl. Without the use of either, infection could not be achieved.

TABLE 1
Summary of observations for L. intracellularis propagation
with or without the use of hydrogen purging.

| | Passage | Bacteria in the | Monolayer infection by | Re-infection |
|---|---|---|---|---|
| | number | Supernatant | immunoperoxidase staining | and Split ratio |
| Treatment 1 | P18 | Positive | 60-70% | 1:1 |
| (hydrogen) | P19 | | ≧80% | 1:3 |
| | P20 | Highly positive | Not done | 1:1.5 |
| | P21 | | Not done | 1:1 |
| Treatment 2 | P18 | Slightly positive | Little or no infection | 1:1 |
| (cysteine | P19 | | 20% | 1:1 |
| chloride) | P20* | | 30-40% (larger foci of | 1:1 |
| | | | infection) | |
| | P21 | Highly positive | Not done | 1:2 |
| Treatment 3 | P18 | Negative | Little or no infection | 1:1 |
| (none) | P19 | | Little or no infection | 1:1 |
| | P20 | | No noticeable infection | 1:1 |
| | P21 | | No infection | Propagation |
| | | | | discontinued |

*Received only cysteine HCl at a final concentration of 0.02%; discontinued the addition of β-NAD and phoshatidylcholine.

In a separate set of experiments, L. intracellularis isolate VP1 (passage 23) was used to infect McCoy cell monolayers. The cells were then placed in a microaerophilic environment for 15 days. At that time, many dead cells were visible in the media, and L. intracellularis organisms in the media did not appear active. Flasks were re-fed with fresh media. The following day, it was noted that the monolayer had begun to detach from the flask, and individual bacteria still did not appear active. Monolayer cells from the flask were lysed using water. Briefly, media from the flask was removed, and approximately 16 ml of sterile de-ionized water was added to the flask, followed by incubation at 37° C. for 10 min. Microscopic examination of the flask revealed swollen and porous McCoy cells. The flask was then gently tapped against the palm of the hand, and a drop of the lysed material was observed under a microscope. No intact cells were observed, indicating a complete lysis of the monolayer. A 4.25% NaCl solution was immediately added to bring the media to near normal physiological osmolarity.

The lysed material was centrifuged at 3200×g for 15 minutes. The resultant pellet was re-suspended in 41 ml of DMEM with 7% FBS, and was used to infect fresh McCoy cells. 35 ml of this lysed L. intracellularis infective material was added to one 175 cm² flask seeded with McCoy cells, and 0.5 ml to one seeded Trac bottle. Both the flask and the Trac bottle were evacuated with a vacuum at 15 psi of Hg, and then purged with pure hydrogen. 5 ml and 0.5 ml of the material were also used to infect McCoy cells seeded in a 25 cm² flask and a Trac bottle, respectively. Following evacuation with 15 psi of vacuum and a nitrogen flush, a 5× stock solution of cysteine HCl was added to the 25 cm² flask and the corresponding Trac bottle to a final concentration of 0.02%. All flasks and Trac bottles were incubated at 37° C. in an incubator containing 8.0% oxygen, 8.8% carbon dioxide and 83.2% nitrogen. Re-feeding was carried out as described above; the flask and Trac bottle which received cysteine HCl were also refed to the same final concentration (0.02%).

The infection was monitored on day 6 or 7 post-infection by immunoperoxidase staining of the corresponding cover slips as described above. Infection was also monitored by flourescent antibody staining. Briefly, cell lysate was collected on a Cytospin microscopic slide using a Cytofunnel sample chamber (Shandon Inc.; Pittsburg, Pa.) and centrifugation for 10 min at 1500 rpm. The sample was air dried and fixed in acetone for 30 sec. The smear was then covered with a 1:400 dilution of rabbit anti-L. intracellularis polyclonal serum and incubated at 37° C. for 30 min. The slide was washed gently with PBS, and a 1:20 dilution of fluorescein-labeled Goat anti-rabbit IgG (H+L; Kirkegaard & Perry Labs) was added to the smear, and the slide was incubated at 37° C. for 30 min. It was then observed under a fluorescent microscope.

These experiments indicate that the L. intracellularis infection of McCoy cells supplemented with cysteine HCl is enhanced compared to infection using hydrogen gassing (Table 2). It was also demonstrated that viability could be restored to L. intracellularis with the use of cysteine HCl. That activity/motility of the bacteria increased in the flask was supported by elevated flagellar expression, as demonstrated by staining of the flagella with fluorescent antibody.

TABLE 2
Summary of observations for L. intracellularis, swine isolate VP1,
on McCoy cells with hydrogen purging or cysteine hydrocholride.

| | Passage | Bacteria in the | Monolayer infection by | Re-infection |
|---|---|---|---|---|
| | number | Supernatant | staining | and Split ratio |
| Treatment 1 | P24 | | Not done | 1:1 |
| (hydrogen) | P25 | Slightly positive, | Lesser infection (smaller and | 1:1 |
| | | Normal motilty | infrequent foci of infection than | |
| | | | Treatment 2). | |
| | P26 | | | Discontinued |
| | | | | in order to |
| | | | | infect roller |
| | | | | bottle |
| Treatment 2 | P24 | | Not done | 1:1 |
| (cysteine | P25 | Slightly positive, | Significantly greater percent of | 1:1 |
| chloride) | | highly motile | infection with larger foci of infection | |
| | | | than Treatment 1 (30% infection). | |
| | | | Lysed material stained by FITC | |
| | | | showed large numbers of flagella. | |
| | P26 | | 50-60% infection | 1:1 |

### EXAMPLE 2

### Propagation of L. intracellularis Hamster Isolate STR: Cysteine Hydrochloride vs. Hydrogen Gassing

L. intracellularis hamster isolate STR (passage 44) was used to infect McCoy cells as follows: McCoy cells were seeded at 2×10⁵ cells per 25 cm² flask in DMEM with 7% FBS and at a comparable density of 8×10³ cells into 48-well plates for monitoring the infection. The cells were incubated overnight at 37° C. in 5% CO₂, and were infected the next day with ˜5×10⁵ bacteria per 25 cm²flask. The infected flasks and the 48-well plates were evacuated at 15 psi of Hg vacuum and purged with hydrogen. A second set of flasks and corresponding Trac bottles were evacuated and purged with pure nitrogen, and cysteine hydrochloride was added to a final concentration of 0.02%. The infected flasks and plates were incubated in a bi-gas incubator at 8.0% CO₂, 8.8% O₂ and 83.2% N₂. The flasks were re-fed with 50% volume of media on day 2 and day 5 post-infection, using DMEM with 5% FBS. A determination of the degree of infection was made on day 5 using immunoperoxidase staining. Lysis and re-infection were carried out based on the extent of infection as monitored by immunostaining of 48-well plate infections (routinely on day 7 post-infection). The media supernatant was collected from the flasks and centrifuged at 3500 g for 30 minutes. The supernatant was discarded and the pellet resuspended in SPG with 5% FBS. The monolayers were washed with PBS, followed by the addition of water and incubation at 37° C. for 15 min, at which time cells began detaching from the flask. The cells were then lysed by passaging 4 to 5 times through a syringe fitted with an 18 ga needle. The lysate was centrifuged at 350 g for 5 min to pellet the cell nuclei. The lysate supernatant was collected and combined with the resuspended pellet from the media supernatant; this was then used to infect a fresh monolayer of McCoy cells.

On day 5 post-infection, cover slips from both the Trac bottles with hydrogen and with cysteine hydrochloride indicated about 20% infection. Thus, cysteine hydrochloride was as effective a supplement for supporting L. intracellularis growth as was hydrogen gassing.

In a similar set of experiments, two 25 cm² flasks were seeded with 2×10⁵ McCoy cells in DMEM with 7% FBS, and incubated overnight at 37° C. in 5% CO₂. Two 48-well plates were also seeded with McCoy cells at a comparable density. The following day, the flasks were infected using the supernatant and cell lysate from L. intracellularis (STR isolate)-infected McCoy cells at passage 49, as described earlier. One flask was evacuated, purged with hydrogen, and placed in an incubator. The second flask was supplemented with 0.02% cysteine hydrochloride (no hydrogen purge) and placed in the bi-gas incubator. The two 48-well plates were infected with L. intracellularis in parallel to the two flasks, and served as controls for monitoring the infection and for immunostaining. Both the flasks and the 48-well plates were incubated in the incubator at 8.0% CO₂, 8.8% O₂ and 83.2% N₂. The flasks were re-fed with 50% volume of media on day 2 and day 5 post-infection using DMEM with 5% FBS.

The bacteria were passaged by cell lysis. Supernatants and cell lysates were prepared from both flasks, as described earlier, and were used to infect fresh monolayers of McCoy cells seeded into 25 cm² or 75 cm² flasks at split ratios of 1:1 or 1:2, depending on the extent of infection as determined by immunostaining of the 48-well plate monolayers. The propagation of L. intracellularis with hydrogen gassing or with cysteine HCl (in the absence of hydrogen gassing) was compared for more than ten passages by assessing the split ratios and by evaluating immunostaining of the monolayers in the 48-well plates. After 10 passages, the culture being propagated using hydrogen gassing had been expanded from one 25 cm² flask to four 25 cm² flasks, a 4-fold increase. The culture propagated using cysteine hydrochloride had been expanded from one 25 cm² flask to twelve 25 cm² flasks, a 12-fold increase. Thus, cysteine hydrochloride can be substituted for hydrogen gassing during L. intracellularis propagation, resulting in comparable or superior bacterial yields.

The 48-well plates infected with or without cysteine hydrochloride were fixed with 80% acetone on day 5 post-infection, and stained using the immunoperoxidase and immunofluorescence methods as described previously. The percent of McCoy cells infected using hydrogen gassing versus cysteine HCl was evaluated after each bacterial passage. Again, these data indicate that cysteine hydrochloride can be substituted for hydrogen gassing during L. intracellularis propagation.

### EXAMPLE 3

### Cultivation of L. intracellularis Swine Isolate PHE/MN-001

Cysteine hydrochloride was also compared to hydrogen gassing for facilitating cultivation of an additional L. intracellularis isolate, PHE/MN-001, in McCoy cell monolayers, as well as the combined effect of using hydrogen gassing and cysteine hydrochloride.

Two 75 cm² flasks were seeded in a similar manner as described for the hamster STR isolate, starting with 6×10⁵ McCoy cells in DMEM with 7% FBS, and incubated overnight at 37° C. in 5% CO₂. Two 48-well plates were seeded with McCoy cells at a comparable density. The following day, the flasks were infected using the supernatant and cell lysate from L. intracellularis (PHE/MN-001 isolate)-infected McCoy cells at passage 40. One flask was evacuated, supplemented with 0.02% cysteine hydrochloride, purged with hydrogen, and placed in the incubator. The second flask was supplemented with 0.02% cysteine hydrochloride and placed in the incubator without first purging using hydrogen. The two 48-well plates were infected with L. intracellularis in parallel with the two flasks, and once again served as controls for monitoring the infection and for immunostaining. The flasks and 48-well plates were incubated in the incubator at 8.0% CO₂, 8.8% O₂ and 83.2% N₂. The flasks were re-fed with 50% volume of media on day 2 and day 5 post-infection using DMEM with 5% FBS.

The bacteria were passaged by cell lysis, as described in earlier. The supernatant and cell lysate were prepared from each flask and used to infect fresh monolayers of McCoy cells seeded into 25 cm² or 75 cm² flasks at split ratios of 1:2 and 1:4. The propagation of L. intracellularis with cysteine hydrochloride and hydrogen gassing or with cysteine HCl alone (in the absence of hydrogen gassing) was compared for more than 9 passages by assessing the expanded flasks, and by evaluating immunostaining of the monolayers in the 48-well plates. After 9 passages, the bacterial counts obtained in the supernatants were similar, regardless of whether in the presence or absence of hydrogen gassing (Table 3). The percent of McCoy cells infected with cysteine hydrochloride and hydrogen gassing, as compared with cysteine HCl alone, was evaluated after each bacterial passage. These results again demonstrate that cysteine hydrochloride can be used as a substitute for hydrogen gassing during L. intracellularis infection and propagation.

TABLE 3
Summary of observations for L. intracellularis,
swine isolate PHE/MN-001 (p49) on McCoy cells with
hydrogen purging or cysteine hydrocholride.

| | | Bacterial Counts |
|---|---|---|
| H₂/Cys HCl | Split Ratio | (per ml supernatant) |
| H₂ + Cys HCl | 1:2 | 5.9 × 10⁷ |
| H₂ + Cys HCl | 1:4 | 4.1 × 10⁷ |
| Cys HCl | 1:2 | 8.5 × 10⁷ |
| Cys HCl | 1:4 | 5.8 × 10⁷ |

1. A method for cultivating Lawsonia intracellularis, comprising culturing cells infected with Lawsonia intracellularis in the absence of molecular H₂ and in the presence of a reducing agent other than molecular H₂.

2. The method of claim 1, wherein said reducing agent other than molecular H₂ is an organic or inorganic reducing agent with a redox potential range of +300 mV to −600 mV.

3. The method of claim 2, wherein said reducing agent is an organic reducing agent.

4. The method of claim 3, wherein said organic reducing agent is selected from the group consisting of: alkyl thiols, aryl thiols, L-cysteine, D-cysteine, homocysteine, β-mercaptoethanol, ethanethiol, propanethiol, dithiothreitol, cysteamine, cysteine persulfides, glutathione, dimercaptosuccinic acid (DMSA), tris(2-carboxyethyl) phosphine hydrochloride (TCEP), tributylphosphine (TBP), and enantiomers, racemic forms or mixtures, free base forms, hydrochlorides, hydrates, and salts thereof.

5. The method of claim 4, wherein said reducing agent is any form of cysteine or a salt thereof.

6. The method of claim 2, wherein said reducing agent is an inorganic reducing agent.

7. The method of claim 6, wherein said inorganic reducing agent is selected from the group consisting of: hydrosulfite (dithionite), thiosulfate, disulfite (metabisulfite), hydrogen sulfide and free base forms, hydrochlorides, hydrates, and salts thereof.

8. The method of claims 1-7 wherein the concentration of said reducing agents are selected from the following ranges: the range of redox potentials are about: +300 mV to −600 mV (mV is milliVolt), more preferred is +100 mV to −400 mV and most preferred is −100 mV to −300 mV, alternatively the reducing agent concentration is in the following ranges, about: 0.8% to 0.0008%, more preferred is 0.4% to 0.004%, more preferred is 0.10% to 0.002% and most preferred 0.02 to 0.002%, alternatively the concentration ranges in milliMolar (tnM), considered especially useful concentration ranges are, about: 0.05 mM to 50.0 mM, more preferred is 0.10 mM to 10.0 mM and most preferred is 0.10 mM to 1.0 mM.

9. The method of claims 1-8, wherein the cells are cultured at an O₂ concentration in the range of about 2% to 18%.

10. A method for cultivating Lawsonia intracellularis, comprising culturing cells infected with Lawsonia intracellularis in the presence of molecular H₂ and at least one organic or inorganic reducing agent other than molecular H₂.

11. The method of claim 10, wherein said reducing agents are organic or inorganic reducing agent, optionally selected from the reducing agents and in such ranges as described in claims 2-8.

12. The method according to claim 11, wherein the cells are cultured at an O₂ concentration in the range of 2% to 18%.

13. A vaccine composition that is effective in treating or preventing a disease in an animal caused by L. intracellularis comprising an immunologically effective amount of L. intracellularis grown by any of the methods of claims 1-12, optionally containing an adjuvant, optionally used to treat a pig.

14. A method of diagnosing a disease in an animal caused by L. intracellularis comprising detecting the presence of antibodies in a sample from said animal that are reactive with or with an antibody generated, or a polynucleotide isolated from the L. intracellularis cultured according to the procedures described herein.

15. A kit useful for diagnosing a disease in an animal caused by L. intracellularis, wherein said kit comprises the L. intracellularis described herein, or a polypeptide or polynucleotide isolated therefrom, or an antibody generated against the L. intracellularis when the L. intracellularis is cultured according to the procedures described herein.
